(19) United States
(12) Patent Application Publication
(10) Pub. No.: US 2021/0353758 A1
(43) Pub. Date: Nov. 18, 2021

(54) MICROCARRIER FOR EMBOLIZATION AND PREPARATION METHOD THEREFOR

(71) Applicants: INDUSTRY FOUNDATION OF CHONNAM NATIONAL UNIVERSITY, Gwangju (KR); KOREA INSTITUTE OF MEDICAL MICROROBOTICS, Gwangju (KR)

(72) Inventors: Jongoh PARK, Gyeonggi-do (KR); Eunpyo CHOI, Gwangju (KR); Chang-Sei KIM, Gwangju (KR); Gwangjun GO, Gwangju (KR); Jiwon HAN, Gwangju (KR)

(21) Appl. No.: 17/286,925

(22) PCT Filed: Jan. 29, 2020
(86) PCT No.: PCT/KR2020/001379

§ 371 Date: Apr. 20, 2021

(30) Foreign Application Priority Data
Feb. 19, 2019 (KR) 10-2019-0019445

(51) Int. Cl. A61K 47/34; A61K 9/16; A61K 31/704; A61K 31/7068; A61K 33/243; A61K 31/282; A61K 31/675; A61K 31/7048; A61K 31/138; A61K 38/14; A61K 31/475; A61K 47/32; A61K 9/113

(52) U.S. Cl. CPC A61K 47/34; A61K 9/1611; A61K 31/704; A61K 31/7068; A61K 33/243; A61K 31/282; A61K 9/113; A61K 31/7048; A61K 31/138; A61K 38/14; A61K 31/475; A61K 47/32; A61K 31/675

(57) ABSTRACT

The present disclosure relates to a microcarrier for embolization, and a preparation method therefor, wherein the microcarrier comprises a biodegradable porous polymer, a stimulus-responsive polymer captured in the biodegradable porous polymer, and drug-supported magnetic nanoparticles captured in the stimulus-responsive polymer, thereby being capable of operating in an in vivo tumor-targeting manner and releasing, by an external stimulus, the drug-supported nanoparticles, so as to be effectively usable in tumor embolization.

### CROSS-REFERENCE TO RELATED APPLICATIONS

This application is a national phase application of PCT Application No. PCT/KR2020/001379, filed on Jan. 29, 2020, which claims the benefit and priority to Korean Patent Application No. 10-2019-0019445, filed on Feb. 19, 2019. The entire disclosures of the applications identified in this paragraph are incorporated herein by references.

### TECHNICAL FIELD

The present disclosure was made with the support of the Ministry of Health and Welfare, Republic of Korea, under Project No. HI19C0642, which was conducted in the research project named “R&D Center for Practical Medical Microrobot Platform” in the research program titled “Korea Health Technology R&D Project” by the Korea Institute of Medical Microrobotics, under management of the Korea Health Industry Development Institute, from 12 Jun. 2019 to 31 Dec. 2022.

The present disclosure relates to a microcarrier for embolization and a preparation method therefor.

### BACKGROUND ART

Embolization refers to a treatment for blocking bloodstream directed toward a specific region in the body and is used as a method for treatment of cancer by selectively passing and lodging an embolus within an arterial vessel running to the tumor. When an embolus bearing a chemotherapy agent (drug) is introduced into an arterial vessel directed to a tumor and selectively necrotizes cancerous cells, the process is called chemoembolization.

However, when introduced into blood vessels in addition to arterial vessels toward tumor, conventional emboli used in tumor embolization may give rise to a significant side effect. For chemoembolization, conventional emboli having a drug loaded thereto are also poor in drug delivery efficiency due to lack of a targeting function as well as causing a side effect of embolizing arterial vessels other than tumor vessels due to a countercurrent.

Therefore, there is an urgent need for research into a drug carrier for embolization that can target tumors and regulate anticancer agent (drug) release in chemoembolization.

### SUMMARY

### Technical Problem

Leading to the present disclosure, intensive and thorough research, conducted by the present inventors, into a drug-loaded embolus targeting a tumor and regulating drug release, resulted in the finding that a microcarrier fabricated by entrapping drug-loaded magnetic nanoparticles within a stimulus-responsive polymer and then capturing the same with a biodegradable porous polymer can target a tumor and release the drug in a controlled manner.

Therefore, an aspect of the present disclosure is to provide a microcarrier comprising: a biodegradable porous polymer; a stimulus-responsive polymer captured by the biodegradable porous polymer; and drug-loaded magnetic nanoparticles entrapped within the stimulus-responsive polymer.

Another aspect of the present disclosure is to provide a method for preparing a microcarrier.

### Technical Solution

The present inventors have made efforts to search for a drug-loaded embolus capable of targeting a tumor and regulating drug release. As a result, it was found that a microcarrier fabricated by entrapping drug-loaded magnetic nanoparticles within a stimulus-responsive polymer and then capturing the same with a biodegradable porous polymer can target a tumor and release the drug in a controlled manner.

The present disclosure relates to a microcarrier comprising: a biodegradable porous polymer, a stimulus-responsive polymer captured by the biodegradable porous polymer; and drug-loaded magnetic nanoparticles entrapped within the stimulus-responsive polymer, and a preparation method therefor.

Below, a detailed description will be given of the present disclosure.

An aspect of the present disclosure pertains to a microcarrier comprising: a biodegradable porous polymer; a stimulus-responsive polymer captured by the biodegradable porous polymer; and drug-loaded magnetic nanoparticles entrapped within the stimulus-responsive polymer.

As used herein, the term “biodegradable” refers to pertaining to degradation by hydrolysis and/or oxidation or through enzymatic activity or microbial activity such as by bacteria, yeasts, fungi, and algae, within a suitable period of time.

As used herein, the term “porous” refers to pertaining to an arrangement of pores, channels, and cages, which may be arranged irregularly, or regularly or periodically. In addition, the pores or channels may be separated from each other or interconnected to each other and may be of one-, two-, or three-dimensional organization.

So long as it forms a porous structure, any biodegradable porous polymer may be used in the present disclosure without particular limitations to kinds thereof. In a particular embodiment of the present disclosure, the biodegradable porous polymer may include a natural or a synthetic polymer.

The natural polymer useful in the present disclosure may be collagen, hyaluronic acid, gelatin, or chitosan, but is not limited thereto.

In the present invention, the synthetic polymer may be PLGA (poly(lactic-co-glycolic acid)), PGA (poly(glycolic acid)), PLA (poly(lactic acid)), or PEG (Polyethylene glycol), but is not limited thereto.

Having a porous form, the biodegradable porous polymer in the present disclosure allows the drug-loaded magnetic nanoparticles entrapped within the polymer to be effectively loaded into pores, channels, and/or cages.

As used herein, the term “stimulus-responsive” refers to pertaining to reaction in response to various in vivo and/or ex vivo stimuli such as temperature, pH, magnetic field, etc.

Moreover, the term “stimulus-responsive polymer”, as used herein, refers to a polymer that can be resolved/degraded in response to various in vivo and/or ex vivo stimuli such as temperature, pH, magnetic field, etc.

In the present disclosure, the stimulus-responsive polymer may include gelatin, PCL (polycaprolactone), chitosan, PNIPAAm (poly(N-isopropylacrylamide)), and/or HEMA (2-hydroxyethyl(methacrylate)), but is not limited thereto.

In the present disclosure, the stimulus-responsive polymer is dissolved/degraded within the body temperature range of 36 to 40° C. and thus can easily release the drug-loaded magnetic nanoparticles to be described below, from the microcarrier.

In the present disclosure, the stimulus-responsive polymer may range in diameter from 1 nm to 1000 μm. For example, the stimulus-responsive polymer is captured by the biodegradable porous polymer, with the drug-loaded magnetic nanoparticles entrapped therein. Thus, the stimulus-responsive polymer may be smaller in diameter than the microcarrier and larger than the drug-loaded magnetic nanoparticles.

In the present disclosure, the stimulus-responsive polymer may be captured in an emulsion form by the biodegradable porous polymer.

As used herein, the term “emulsion” refers to a mixture of two or more liquids that cannot be immiscible with each other by a general method. Various types of emulsions can be prepared by mixing two or more liquids. For example, the emulsion may be an oil-in-water emulsion in which oil is a dispersed phase, with water serving as a dispersion medium, or a water-in-oil emulsion in which water is a dispersed phase, with oil serving as a dispersion medium. Alternatively, the emulsion may be a water-in-oil-in-water type in which a water-in-oil emulsion exists as a dispersed phase in the dispersion medium of water, or an oil-in-water-in-oil type in which an oil-in-water emulsion exists as a dispersed phase in the dispersion medium of oil, but without limitations thereto.

According to an embodiment of the present disclosure, the microcarrier may be in a structure where the stimulus-responsive polymer in a water phase is captured by the biodegradable porous polymer in an oil phase or where the stimulus-responsive polymer in an oil phase is captured by the biodegradable porous polymer in a water phase.

The term “magnetic nanoparticles”, as used herein, means nanoparticles that have magnetic sensitivity, with magnet contained therein. They may be made of various materials.

Any magnetic nanoparticles that are of magnetic sensitivity can be used in the present disclosure, with no particular limitations to concrete types thereof. However, they may be made of a magnetic material or a magnetic alloy.

The magnetic material usable in the present disclosure may be Fe, Co, Mn, Ni, Gd, Mo, MM′₂O₄, or MₓO_{y}, but is not limited thereto.

In the present disclosure, the magnetic material may be MM′₂O₄ or MₓO_{y} wherein M and M′ are each independently Fe, Co, Ni, Mn, Zn, Gd, or Cr, x is an integer of 1 to 3, and y is an integer of 1 to 5, but with no limitations thereto.

The magnetic alloy usable in the present disclosure may be CoCu, CoPt, FePt, CoSm, NiFe, or NiFeCo, is not limited thereto.

Having magnetic sensitivity, the magnetic nanoparticles used in the present disclosure allow the microcarrier of the present disclosure to precisely target a tumor site through magnetic field control.

In the present disclosure, the microcarrier targeted at a tumor site embolizes an arterial vessel running to the tumor, after which the stimulus-responsive polymer is dissolved to release the magnetic nanoparticles.

According to the present disclosure, the magnetic nanoparticles may range in diameter from 1 to 1,000 nm, from 1 to 900 nm, from 1 to 800 nm, from 1 to 700 nm, from 1 to 600 nm, from 10 to 1,000 nm, from 10 to 900 nm, from 10 to 800 nm, from 10 to 700 nm, from 10 to 600 nm, from 50 to 1,000 nm, from 50 to 900 nm, from 50 to 800 nm, from 50 to 700 nm, from 50 to 600 nm, for example, from 50 to 500 nm. Magnetic nanoparticles with a diameter greater than 1,000 nm may be poor in bioavailability, e.g., the nanoparticles may block blood vessels when introduced into the body.

In the present disclosure, the magnetic nanoparticles may be coated with a surface coating agent to lower toxicity and increase a rate of reaching a lesion.

In the present disclosure, the surface coating agent may be at least one selected from the group consisting of starch, polyethylenimine, dextran, citrate, carboxydextran, PEG (polyethyleneglycol), and derivatives thereof, but is not limited thereto.

In the present disclosure, the drug may be a protein, a peptide, a vitamin, a nucleic acid, a synthetic drug, or a natural extract, but is not limited thereto.

In the present disclosure, the synthetic drug may be at least one selected from the group consisting of doxorubicin, epirubicin, gemcitabine, cisplatin, carboplatin, procarbazine, cyclophosphamide, dactinomycin, daunorubicin, etoposide, tamoxifen, mitomycin, bleomycin, plicamycin, transplatinum, vinblastine, and methotrexate, but is not limited thereto.

In the present disclosure, the drug loaded onto the magnetic particles penetrates into cancer cells in a tumor and then can be selectively released from the magnetic nanoparticles in response to an external stimulus.

In the present disclosure, the external stimulus may include a near infrared (NIR) radiation, an ultrasonic wave, and/or an AC magnetic field, but is not limited thereto.

In the present disclosure, the microcarrier may 40 to 1000 μm in diameter. Given a diameter less than 40 μm, the microcarriers may flow into arterial vessels running to a site other than a tumor and may be distributed in other organs. Microcarriers with a diameter exceeding 1,000 μm are difficult to inject through a catheter tube.

In the present disclosure, the microcarrier may further comprise a pharmaceutically acceptable excipient, for example, a diluent, a release retardant, an inactive oil, and/or a binder, but without limitations thereto.

Another aspect of the present disclosure pertains to an anticancer pharmaceutical composition comprising the microcarrier.

In the present disclosure, the cancer may be liver cancer, breast cancer, stomach cancer, lung cancer, prostate cancer, ovarian cancer, bronchial cancer, nasopharyngeal cancer, larynx cancer, pancreatic cancer, bladder cancer, colorectal cancer, uterine cervical cancer, or thyroid cancer, but is not limited thereto.

In the present disclosure, the pharmaceutical composition may be used for tumor embolization. Therefore, the pharmaceutical composition can be easily used in vessel embolization for tumor therapy.

According to an embodiment of the present disclosure, the pharmaceutical composition may be used for transarterial chemoembolization.

The pharmaceutical composition of the present disclosure may comprise a pharmaceutically acceptable carrier.

According to an embodiment of the present disclosure, the pharmaceutically acceptable carrier may be one typically used for formulation and examples thereof include lactose, dextrose, sucrose, sorbitol, mannitol, starch, acacia gum, calcium phosphate, alginate, gelatin, calcium silicate, microcrystalline cellulose, polyvinylpyrrolidone, cellulose, water, syrup, methyl cellulose, methyl hydroxy benzoate, propyl hydroxy benzoate, talc, magnesium stearate, and mineral oil, but are not limited thereto.

In addition to the above ingredients, the pharmaceutical composition of the present disclosure may further contain a lubricant, a humectant, a sweetener, a flavorant, an emulsifier, a suspending agent, a preservative, etc.

For oral administration, a solid formulation of the pharmaceutical composition of the present disclosure may include a tablet, a pill, a powder, a granule, a capsule, etc. Such a solid formulation may be prepared by mixing the ingredient with one or more excipients, for example, starch, calcium carbonate, sucrose, lactose, gelatin, etc. In addition to the simple excipient, a lubricant such as magnesium stearate, talc, etc. may be employed.

A liquid formulation for oral administration of the pharmaceutical composition according to the present disclosure may be exemplified by a suspension, a solution, an emulsion, a syrup, and so on, and may comprise various excipients, for example, wetting agents, sweeteners, flavors, preservatives, etc. in addition to commonly used simple diluents such as water and liquid paraffin.

Formulations for parenteral administration of the pharmaceutical composition according to the present disclosure may be exemplified by sterilized aqueous solutions, non-aqueous solvents, suspensions, emulsions, lyophilized pellets, suppositories, etc.

In the present disclosure, non-aqueous solutions and suspensions may contain propylene glycol, polyethylene glycol, vegetable oil such as olive oil, and injectable esters such as ethylolate, but with no limitations thereto.

In the present disclosure, the injection may include a typical additive, such as a solubilizer, an isotonic agent, a suspending agent, an emulsifier, a stabilizer, a preservative, and so on, but without limitations thereto.

A suitable dose of the pharmaceutical composition according to the present disclosure may vary depending on various factors including pharmaceutical formulation methods, administration methods, the patient's age, body weight, sex, severity of diseases, diet, administration time, administration route, an excretion rate, and sensitivity for a used pharmaceutical composition. Physicians with average skill may easily determine and prescribe dosage levels effective for treating or preventing target disorders or diseases. The pharmaceutical composition of the present disclosure may be administered at a daily dose of 0.001 to 10000 mg/kg.

The pharmaceutical composition of the present disclosure may be formulated as general formulations using the pharmaceutically acceptable carriers and/or excipients according to methods easily practiced by a person skilled in the art to which the present disclosure pertains, to be prepared as a unit dosage form or to be prepared by introducing the composition into a multi-dosage container. The general formulation refers to a solution in oil or aqueous medium, a suspension, an emulsion, an extract, a powder, a granule, a tablet, or capsule, and may further contain a dispersant or a stabilizer.

Another aspect of the present disclosure pertains to a method for treatment of cancer, the method comprising a step of administering the microcarrier to a subject in need thereof.

The method for treatment of cancer according to the present disclosure uses the anticancer pharmaceutical composition comprising the microcarrier according to the present disclosure. Thus, descriptions of common contents therebetween are omitted to avoid excessive complexities.

As used herein, the term “administering” is intended to refer to the provision of a substance of interest in a suitable manner into a patient. So long as it allows the pharmaceutical composition of the present disclosure to reach a target tissue, any administration route may be taken. The pharmaceutical composition may be administered orally or parenterally. In addition, the composition of the present disclosure may be administered with the aid of a device for guiding the active ingredient to target cells.

In the present disclosure, the “subject” is not particularly limited, but may include, for example, a human, a monkey, a cow, a horse, sheep, a pig, a chicken, a turkey, a quail, a cat, a dog, a mouse, a rat, a rabbit, or a guinea pig.

Another aspect of the present disclosure pertains to a use of the microcarrier for treatment of cancer.

Another aspect of the present disclosure pertains to a method for preparing a microcarrier, the method comprising the following steps:

a first loading step of loading a drug onto magnetic nanoparticles;

a second loading step of loading magnetic nanoparticles into a stimulus-responsive polymer; and

a third loading step of loading the stimulus-responsive polymer into a biodegradable porous polymer.

In the present disclosure, the first loading step may further comprise a step of coating the magnetic nanoparticles with at least one selected from the group consisting of starch, polyethylenimine, dextran, citrate, carboxydextran, PEG (polyethyleneglycol), and derivatives thereof.

In the present disclosure, the coating step may be carried out prior to or subsequent to loading a drug onto the magnetic nanoparticles, but without limitations thereto.

In the present disclosure, the loading in each loading step is not particularly limited, but may be achieved by mixing each load with each support.

In the present disclosure, the third loading step may be carried out by emulsification using a fluidic device, but without limitations thereto.

As used herein, the term “emulsion” refers to a mixture of two or more liquids that cannot be immiscible with each other by a general method. Various types of emulsions can be prepared by mixing two or more liquids.

In an embodiment of the present disclosure, the emulsion may be an oil-in-water emulsion in which oil is a dispersed phase, with water serving as a dispersion medium, or a water-in-oil emulsion in which water is a dispersed phase, with oil serving as a dispersion medium. Alternatively, the emulsion may be a water-in-oil-in-water type in which a water-in-oil emulsion exists as a dispersed phase in the dispersion medium of water, or an oil-in-water-in-oil type in which an oil-in-water emulsion exists as a dispersed phase in the dispersion medium of oil, but without limitations thereto.

In the present disclosure, the emulsification may be achieved by any method that is typically used to prepare a (multi) emulsion, for example, using a fluidic device to perform mass transfer within flow channels, but with no limitations thereto.

In describing the method for preparation of a microcarrier, the overlapping contents for the microcarrier are omitted in order to avoid excessive complexities.

### Advantageous Effects

The present disclosure relates to a microcarrier and a preparation method therefor. The microcarrier of the present disclosure comprises a biodegradable porous polymer, a stimulus-responsive polymer captured by the biodegradable porous polymer, and drug-loaded magnetic nanoparticles entrapped within the stimulus-responsive polymer and can target a tumor in vivo and release the drug-loaded nanoparticles in response to an external stimulus, thus finding advantageous applications in tumor embolization.

### BRIEF DESCRIPTION OF THE DRAWINGS

FIGS. 1a, 1b and 1c are photographic images of the microcarriers according to an embodiment of the present disclosure, taken by an optical microscope (Eclipse Ti-U, Nikon, Japan).

FIG. 1d is a photographic image of the microcarriers according to an embodiment of the present disclosure, observed with the naked eye.

FIG. 2 shows results of a magnetic operation experiment performed on the microcarriers according to an embodiment of the present disclosure.

FIGS. 3a and 3b show results of an experiment of releasing magnetic nanoparticles from the microcarriers according to an embodiment of the present disclosure.

### BEST MODE FOR CARRYING OUT THE DISCLOSURE

A microcarrier, comprising: a biodegradable porous polymer; a stimulus-responsive polymer captured by the biodegradable porous polymer; and drug-loaded magnetic nanoparticles entrapped within the stimulus-responsive polymer.

### DETAILED DESCRIPTION

Hereinafter, the present disclosure will be described in more detail through examples. The following examples are for illustrative purposes only and it will be apparent to those of ordinary skill in the related art that the scope of this disclosure is not limited by the examples.

### Preparation Example: Preparation of Microcarrier

A. Fabrication of Fluidic Device

Two-way flow channels were constructed by inserting 21G needles into PVC tubes (inner diameter 1/32 inches×outer diameter 3/32 inches) and then equipped with a syringe pump to fabricate a fluidic device for preparation of microcarriers.

B. Preparation of Microcarrier

A PLGA solution containing PLGA (poly(lactic-co-glycolic acid), 70 mg/ml) in 1 ml of DCM/Span80 (100:1, v/v) and a gelatin solution containing gelatin (200 mg/ml) and Fe₃O₄ nanoparticles (fluidMAG-D, Chemicell, Germany; 10 mg/mL) in 100 ml of 1% PVA (polyvinyl alcohol) were prepared. Next, the PLGA solution (1 ml) was mixed with the gelatin solution (0.8 ml) (2,500 rpm, 2.5 min) to give a W-O emulsion which was then poured into a 26G needle syringe and inserted into the center of each of the 21G needles in the fluidic device fabricated above (solution: PVA 1%, flow rate: 3 ml/min). The W-O-W droplets formed in the channels were introduced along the 21G needles in the fluidic device and collected in a deionized water-filled 500-ml beaker in an ice bath. The DCM (dichloromethane) entrapped within the collected W-O-W droplets were evaporated by gently stirring for 6 hours. Finally, the DCM-depleted W-O-W droplets (microcarriers) were washed three times with deionized water and stored in a 25-ml vial containing deionized water.

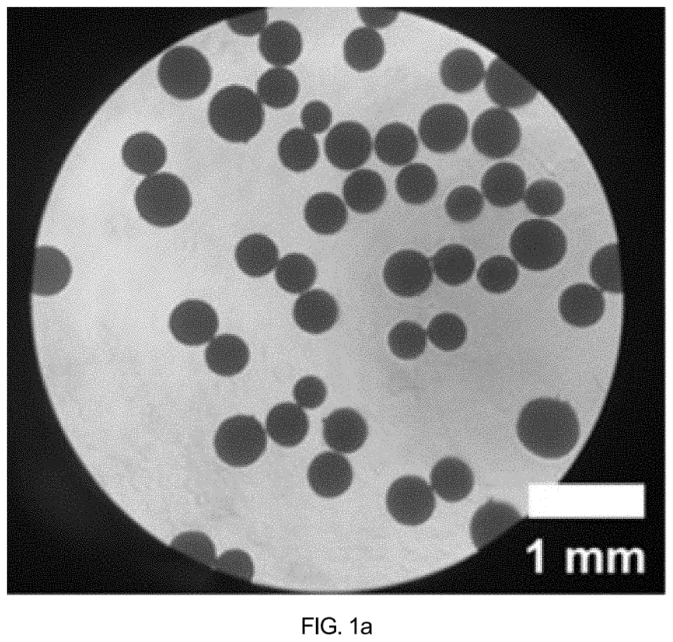

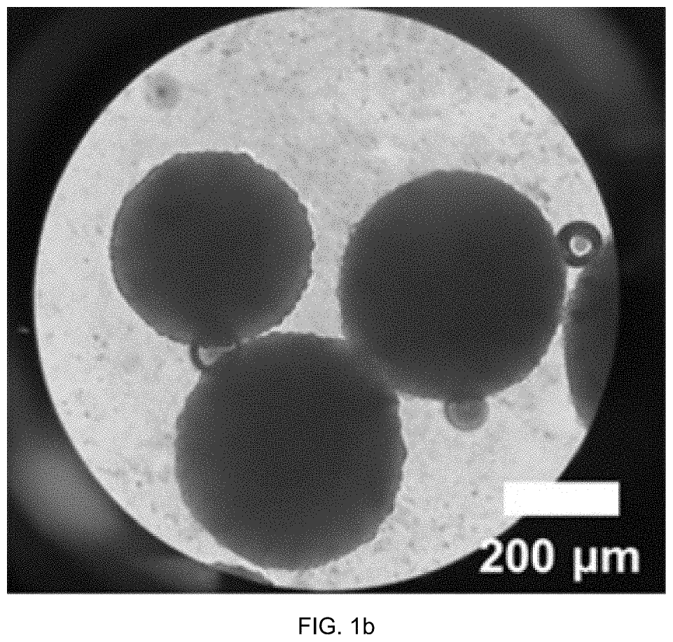

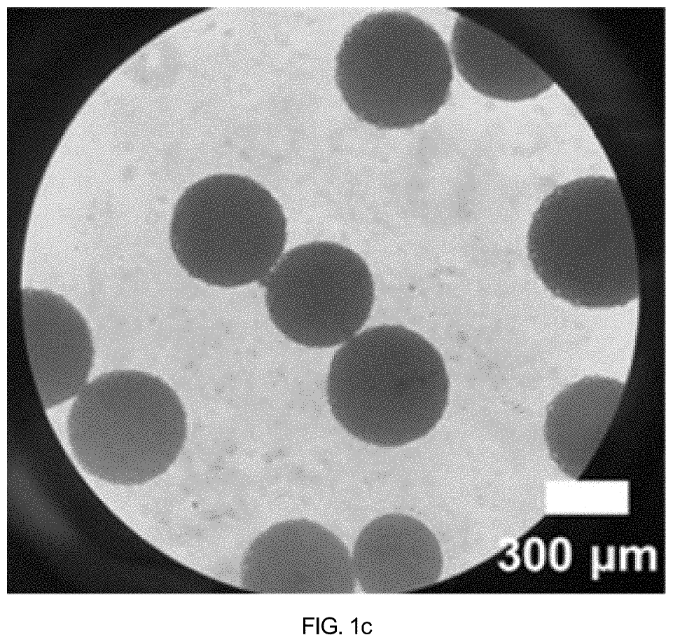

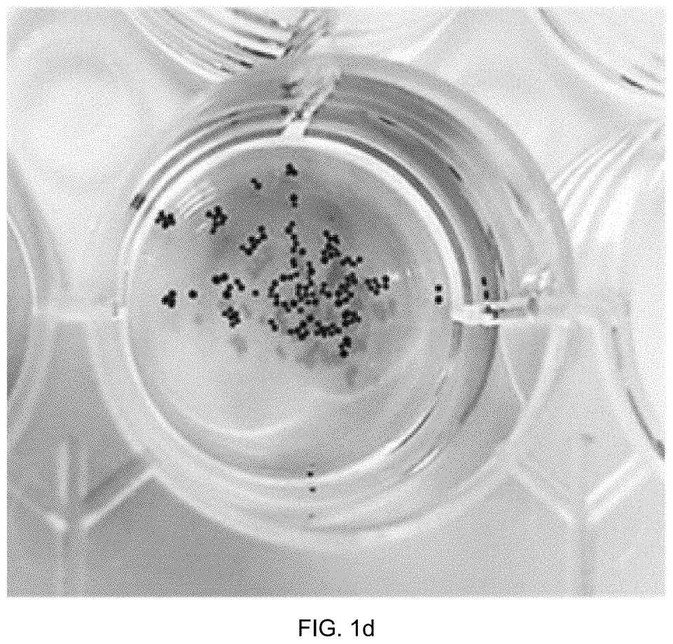

The microcarriers thus prepared were observed under an optical microscope (Eclipse Ti-U, Nikon, Japan) and the results are depicted in FIGS. 1a to 1c. An image observed with the naked eye is given in FIG. 1d.

### Experimental Example 1: Magnetic Operability of Microcarrier

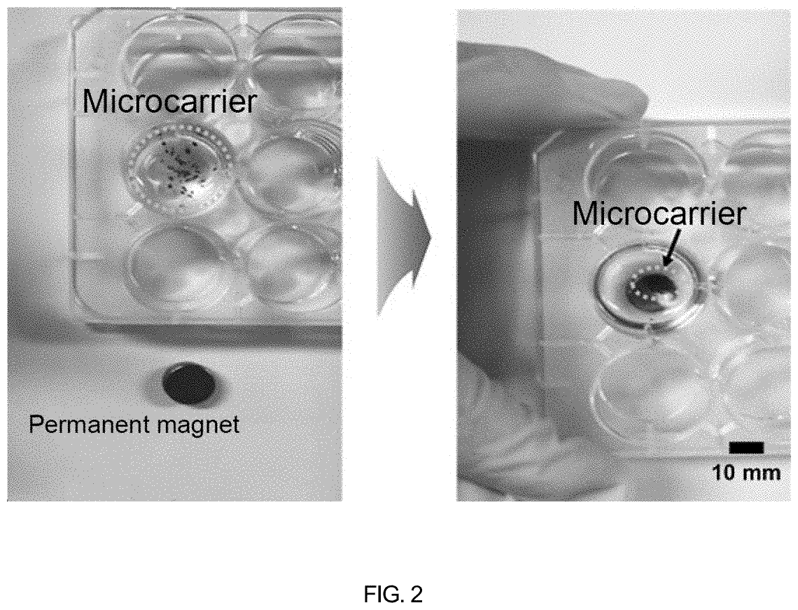

The microcarriers prepared in the Preparation Example were positioned on a 12-well plate and tested for magnetic mobility by using a neodymium permanent magnet (10 mm in diameter and 5 mm in thickness, N35 grade, JL Magnet, Korea). The result is depicted in FIG. 2.

As can be seen in FIG. 2, the microcarriers were attracted toward the permanent magnet by the magnetic field generated by the permanent magnet as the magnet approached the microcarriers.

### Experimental Example 2: Release of Magnetic Nanoparticle from Microcarrier

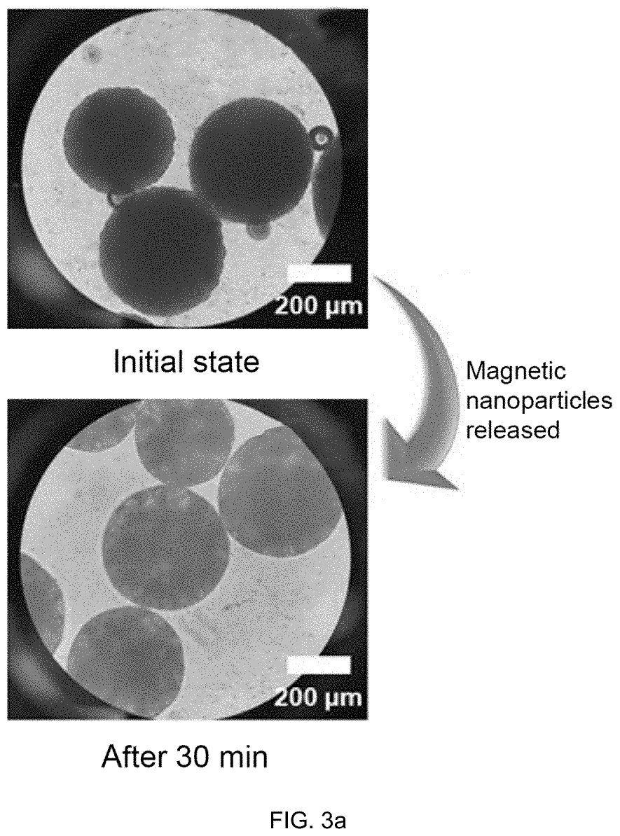

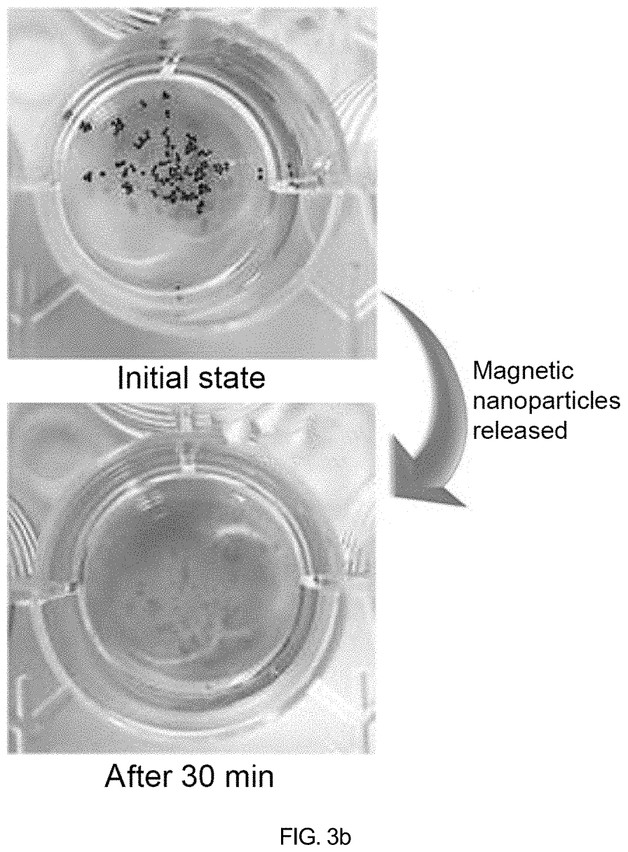

The microcarriers prepared in the Preparation Example were positioned on a 12-well plate and incubated for 30 min in a 37° C. chamber before the release of magnetic nanoparticles was observed by photography (EOS 600D, CANON, Japan) and microscopy (Eclipse Ti-U, Nikon, Japan). The results are depicted in FIGS. 3a and 3b.

As can be seen in FIG. 3a, the PBS solution containing microcarriers did not change in color before temperature stimulation, but underwent a color change after 30 min of temperature stimulation, implying that the stimulus-responsive polymer (gelatin) is dissolved to release the magnetic nanoparticles from the microcarriers.

In addition, as shown in FIG. 3b, there is a difference in the transmittance of the microcarrier before and after temperature stimulation, indicating the release of magnetic nanoparticles from the microcarrier, as well.

### INDUSTRIAL APPLICABILITY

The present disclosure relates to a microcarrier for embolization and a preparation method therefor.

1. A microcarrier, comprising:
a biodegradable porous polymer;
a stimulus-responsive polymer captured by the biodegradable porous polymer; and
drug-loaded magnetic nanoparticles entrapped within the stimulus-responsive polymer.

2. The microcarrier of claim 1, wherein the biodegradable porous polymer is at least one selected from the group consisting of PLGA (poly(lactic-co-glycolic acid)), PGA (poly(glycolic acid)), PLA (poly(lactic acid)), PEG (Polyethylene glycol), collagen, hyaluronic acid, gelatin, and chitosan.

3. The microcarrier of claim 1, wherein the stimulus-responsive polymer is at least one selected from the group consisting of gelatin, PCL (polycaprolactone), chitosan, PNIPAAm (poly(N-isopropylacrylamide)), and HEMA (2-hydroxyethyl(methacrylate)).

4. The microcarrier of claim 1, wherein the magnetic nanoparticles are made from at least one selected from the group consisting of Fe, Co, Mn, Ni, Gd, Mo, MM′₂O₄, MₓO_{y} (M and M′ are each independently Fe, Co, Ni, Mn, Zn, Gd, or Cr, x is an integer of 1 to 3, and y is an integer of 1 to 5), CoCu, CoPt, FePt, CoSm, NiFe, and NiFeCo.

5. The microcarrier of claim 1, wherein the magnetic nanoparticles are coated with a surface coating agent.

6. The microcarrier of claim 5, wherein the surface coating agent is at least one selected from the group consisting of starch, polyethylenimine, dextran, citrate, carboxydextran, PEG (polyethyleneglycol), and derivatives thereof.

7. The microcarrier of claim 1, wherein the drug is at least one selected from the group consisting of doxorubicin, epirubicin, qemcitabine, cisplatin, carboplatin, procarbazine, cyclophosphamide, dactinomycin, daunorubicin, etoposide, tamoxifen, mitomycin, bleomycin, plicamycin, transplatinum, vinblastine, and methotrexate.

8. An anticancer pharmaceutical composition comprising the microcarrier of claim 1.

9. The anticancer pharmaceutical composition of claim 8, wherein the anticancer pharmaceutical composition is for use in tumor embolization.

10. A method for preparation of a microcarrier, the method comprising:
a first loading step of loading a drug onto magnetic nanoparticles;
a second loading step of loading magnetic nanoparticles into a stimulus-responsive polymer; and
a third loading step of loading the stimulus-responsive polymer into a biodegradable porous polymer.

11. The method of claim 10, wherein the third loading step is carried out by emulsification using a fluidic device.
